(19) United States
(12) Patent Application Publication
(10) Pub. No.: US 2024/0009417 A1
(43) Pub. Date: Jan. 11, 2024

(54) SYSTEM, METHOD AND COMPUTER-READABLE MEDIUM FOR IDENTIFYING A GRIP TECHNIQUE BEING APPLIED TO A MASK

(71) Applicant: CAE HEALTHCARE CANADA INC., St-Laurent (CA)

(72) Inventors: Mathieu LEONARD, St-Remi (CA); Laurent DESMET, Toulouse (FR); Mayank SHARMA, Ottawa (CA)

(21) Appl. No.: 18/471,459

(22) Filed: Sep. 21, 2023

Related U.S. Application Data
(63) Continuation of application No. PCT/CA2022/050423, filed on Mar. 22, 2022.
(60) Provisional application No. 63/163,998, filed on Mar. 22, 2021.

(51) Int. Cl. A61M 16/06

(52) U.S. Cl. CPC A61M 16/0605; A61M 2205/583; A61M 2205/3327

(57) ABSTRACT

A non-transitory computer-readable medium, a system and a method are provided for identifying a grip technique being applied to a face mask. The mask is for sealingly engaging a face along a periphery around the nose and mouth. The method comprises the steps of comparing pressure data indicative of a pressure distribution applied along the perimeter with distribution patterns distinctive of different grip techniques; recognizing or detecting which of the grip techniques is being applied on the basis of an outcome of the comparing; and providing an indication of the recognized grip technique to a user interface.

### CROSS REFERENCE TO RELATED APPLICATIONS

This application is a continuation of International Application No. PCT/CA2022/050423, filed Mar. 22, 2022, and which claims priority to U.S. Provisional Application No. 63/163,998, filed Mar. 22, 2021, each of which applications are hereby incorporated by reference in their entirety.

### TECHNICAL FIELD

The technical field generally relates to masks and more particularly to the placement of masks.

### BACKGROUND

Developing or maintaining a proper mask placement technique, such as a Bag-Valve-Mask (BVM) mask, on the face of a human or of a training manikin can be difficult, since there are different techniques to learn, and no data available on these. Applying a proper grip technique on a mask requires a lot of precise maneuvers and skills, to ensure that the edge of the mask is correctly sealed around the nose and mouth and that the airways are properly ventilated.

Current evaluation or training systems for BVM include instrumented masks or manikins which are used to detect and quantify ventilated air volumes, cadence and/or pressure applied on the mask. However, although these systems are configured to gather data that can help clinicians determine whether ventilation is adequate, they do not provide adequate guidance on how to adjust and improve the trainees' maneuvers and grip techniques.

There is thus a need for systems and methods which overcome the limitations of existing training systems.

### BRIEF SUMMARY

According to an aspect, there is provided a system for identifying a grip technique being applied to a face mask for sealingly engaging a face along a mask periphery around the nose and mouth, the system comprising: a processor and a non-transitory computer-readable medium having stored thereon processor-executable instructions for: accessing pressure data indicative of a pressure distribution applied along the mask periphery; comparing the pressure data with pressure distribution pattern data distinctive of different grip techniques; recognizing, detecting or identifying which of the different grip techniques is being applied on the basis of an outcome of the comparing; and providing an indication of the grip technique recognized, or detected, to a user interface.

In some embodiments, the non-transitory computer-readable medium further has stored thereon processor-executable instructions for: generating feedback regarding pressure adjustments to be made for the proper application of the recognized grip technique; and providing the feedback to the user interface.

In some embodiments, the providing the feedback is performed in real time while the face mask is being positioned.

In some embodiments, the user interface comprises a display and providing the indication of the grip technique is performed through a graphical user interface presented within the display.

In some embodiments, the user interface comprises a visual representation of the pressure distribution applied along the periphery of the face mask.

In some embodiments, the visual representation comprises at least one of colours, icons, letters, and numbers.

In some embodiments, the outcome is associated with a probability or likelihood that the pressure distribution applied is one of the pressure distribution patterns distinctive of the recognized grip technique.

In some embodiments, the non-transitory computer-readable medium comprises a trained machine learning model, and wherein recognizing a given one of the different grip techniques comprises predicting the grip technique applied using the trained machine learning model.

In some embodiments, the trained machine learning model comprises a trained machine learning classification model.

In some embodiments, the trained machine learning model is configured to output a performance score indicative of the closeness of the pressure distribution applied on the mask from the pressure distribution pattern associated with the grip technique recognized.

In some embodiments, the trained machine learning model is trained for assigning the pressure data to one of: an E-C grip technique, a two-hand grip technique and a rotated hold technique.

In some embodiments, the trained machine learning model is a support-vector machine model or a neural network model.

In some embodiments, the non-transitory computer-readable medium further has stored thereon processor-executable instructions for generating statistical data from the pressure data, the statistical data including mean, standard deviation, minimum and maximum values for time buffers, and the comparing is based on the statistical data.

In some embodiments, the user interface further comprises an indication of how close the pressure distribution applied is from the pressure distribution pattern distinctive of the recognized grip technique.

In some embodiments, the non-transitory computer-readable medium further comprises processor-executable instructions for monitoring a performance of users in applying a given grip technique, by storing pressure data over time and associated performance scores assigned to the users.

In some embodiments, the system further comprises pressure transducers configured to generate pressure data signals from which the pressure data is derived.

In some embodiments, the pressure transducers are provided at or near the mask periphery.

In some embodiments, the system further comprises a printed circuit board (PCB) provided on the face mask, the PCB comprising: input ports for collecting the pressure data signals from the pressure transducers; and output ports for sending the pressure data and/or the indication of the grip technique recognized via a wired or wireless connection to the computer-readable medium.

In some embodiments, the processor and computer-readable medium are mounted on the PCB.

In some embodiments, the user interface comprises lights provided along the periphery of the face mask, the lights providing assessment regarding pressure adjustments to be made for the recognized grip technique.

In some embodiments, the system further comprises a position sensor provided on the mask for generating position data signals, and wherein the recognizing also takes into account the position data signals.

In some embodiments, the non-transitory computer-readable medium further comprises processor-executable instructions for filtering the pressure data to prevent the comparing from being applied to portions of the pressure data corresponding to placing the face mask on and removing it from the face.

In some embodiments, the non-transitory computer-readable medium further comprises processor-executable instructions for detecting, from the pressure data, transitional periods corresponding to periods during which the face mask is being placed, removed or changed in position, and wherein the filtering is performed to prevent the comparing from being applied to portions of the pressure data generated during the transitional periods.

In some embodiments, the system further comprises a barometric and/or a temperature sensor, wherein the pressure data are adjusted as a function of ambient temperature and/or ambient pressure data generated as the grip technique is being applied, whereby the comparing takes into consideration ambient conditions

In some embodiments, the system further comprises airflow sensors for generating airflow data or signals within the mask portion, wherein the non-transitory computer-readable medium further comprises processor-executable instructions for detecting leaks by comparing the airflow data or signals with reference air flow data associated with an adequate reference sealing engagement of the face mask on the face.

In some embodiments, the system further comprises a training manikin, and wherein the pressure transducers are provided around the nose and mouth of the training manikin.

In some embodiments, the mask is part of a bag-valve-mask.

According to another aspect, there is provided a computer-implemented method for identifying a grip technique being applied to a face mask for sealingly engaging a face along a periphery around the nose and mouth, the method comprising: comparing pressure data indicative of a pressure distribution applied along the periphery with distribution patterns distinctive of different grip techniques; recognizing which of the grip techniques is being applied on the basis of an outcome of the comparing; and providing an indication of the recognized grip technique to a user interface.

In various embodiments, the method can further have any feature or step that the system can implement, as described above, or any combination thereof.

According to yet another aspect, there is provided a non-transitory computer-readable medium having stored thereon processor-executable instructions for identifying a grip technique being applied to a face mask for sealingly engaging a face along a perimeter around the nose and mouth, the instructions causing one or more processors to perform a method comprising: comparing pressure data indicative of a pressure distribution applied along the periphery with distribution patterns distinctive of different grip techniques; recognizing which of the grip techniques is being applied on the basis of an outcome of the comparing; and providing an indication of the recognized grip technique to a user interface.

In various embodiments, the non-transitory computer-readable medium can further have instructions stored thereon for causing a processor to execute steps defined in the previous paragraphs, or any combination thereof.

### BRIEF DESCRIPTION OF THE DRAWINGS

The features and advantages of the present invention will become apparent from the following detailed description, taken in combination with the appended drawings, in which:

FIG. 1A is a representation of a system for identifying a grip technique applied on a face mask, according to a first possible embodiment;

FIG. 1B is a flow chart of the steps of a corresponding method, according to a possible implementation;

FIG. 2 is a schematic illustration of a system for identifying a mask grip technique, according to a second possible embodiment;

FIG. 3 is a schematic illustration of a printed circuit board (“PCB”) used with an instrumented mask, such as the one shown in FIG. 2, according to a possible embodiment;

FIG. 4 is a schematic illustration of components of a system according to a third possible embodiment;

FIG. 5 is a schematic illustration of components of a system according to a fourth possible embodiment;

FIG. 6 is a schematic illustration of components of a system according to a fifth possible embodiment;

FIG. 7 is a schematic illustration of components that can be part of system embodiments presented in FIG. 2, 4, 5 or 6, where the user interface can be provided on the mask and/or as a graphical user interface;

FIG. 8 shows a possible embodiment of a graphical user interface, in which the pressure distribution applied on the periphery of the face mask is represented in real time, along with other collected metrics and manipulation instructions;

FIG. 9 is another possible embodiment of a graphical user interface, in which the pressure distribution applied is represented, along with a reference pressure distribution pattern corresponding to a specific grip technique and manipulation instructions;

FIG. 10 is a flow chart of steps of a method for configuring a system for evaluating the performance of a user in positioning a face mask, according to a possible embodiment;

FIG. 11 is a visual representation of the possible steps of the computer-implemented method of FIG. 10;

FIG. 12A is a schematic illustration showing a user using a “rotated hold” technique on the face mask of a BVM;

FIG. 12B is a schematic illustration showing a user using a “C-E” technique on the face mask of a BVM; and

FIG. 13 is a schematic illustration of a manikin equipped with pressure transducers, placed around the mouth and nose of a manikin.

### DETAILED DESCRIPTION

In the following description, the same numerical references refer to similar elements. In addition, for the sake of simplicity and clarity, namely so as to not unduly burden the figures with several references numbers, not all figures contain references to all the components and features, and references to some components and features may be found in only one figure, and components and features of the present disclosure which are illustrated in other figures can be easily inferred therefrom. The embodiments, geometrical configurations, materials mentioned and/or dimensions shown in the figures are optional and are given for exemplification purposes only.

Furthermore, although the various exemplary embodiments of the system for identifying a grip technique described herein may be used in relation with a bag-valve-mask, it is understood that the system may be useful for other types of masks or devices which require the application of pressure thereon. The term [symbol] mask [symbol] , in the context of the present disclosure, encompass all other types of masks with which the described system could be used and may be useful. In addition, although the optional configurations as illustrated in the accompanying drawings comprise various components, not all of these components and configurations are essential and thus should not be taken in their restrictive sense, i.e., should not be taken as limiting the scope of the present disclosure. The components or method steps of the different embodiments described below can be combined to form other embodiments, according to the present invention.

As will be explained below in relation to various embodiments, a system is provided for recognizing a grip technique being applied to the face mask, which may be part of a bag-valve-mask (BVM), along a periphery or perimeter around the nose and mouth. The system comprises at least a non-transitory computer-readable medium (also referred to as memory or storage medium), and a processor. The computer-readable medium has stored thereon computer-readable instructions and the processor can execute these instructions. The instructions are for processing pressure data to recognize or detect a grip technique, by comparing a pressure distribution with pressure distribution patterns distinctive of different grip techniques. The comparison of the pressure distribution with the different reference pressure distribution patterns can be performed using algorithms with different thresholds, but a machine learning model such as a statistical machine learning model usable as a classifier is preferably used. In possible embodiments, the model can be usable as a probabilistic classifier or as a non-probabilistic classifier of which the output can be transformed into a probability distribution over classes, for instance by using Platt scaling. In possible embodiments, the statistical machine learning model can be a support vector machine (SVM) trained through regression analysis in order to create a set of hyperplanes that can be used as a non-probabilistic classifier, of which the output can be transformed into a probability distribution over classes, for instance by using Platt scaling. The stored instructions also allow the processor to provide an indication of the recognized grip technique to a user interface, based on the outcome of the comparison. The user interface can be of different types, such as lights provided on the face mask or a graphical user interface. The computer-readable medium and processor are part of a processing device, which can take different configurations, such as a PCB, a smart phone or tablet, a laptop, a desktop computer, a single or distributed group of servers. A corresponding method is also provided, where the steps are executed by a processing device. In possible embodiments, pressure transducers, which may be part of pressure sensors, can also be included as part of the system. Pressure transducers are used to detect the pressure being applied on the periphery of the face mask (also referred to hereafter as “mask”) and generate pressure data signals, which can be converted and stored in memory as pressure data. The pressure transducers can be provided at the periphery of the mask of a BVM or on a training manikin, for example.

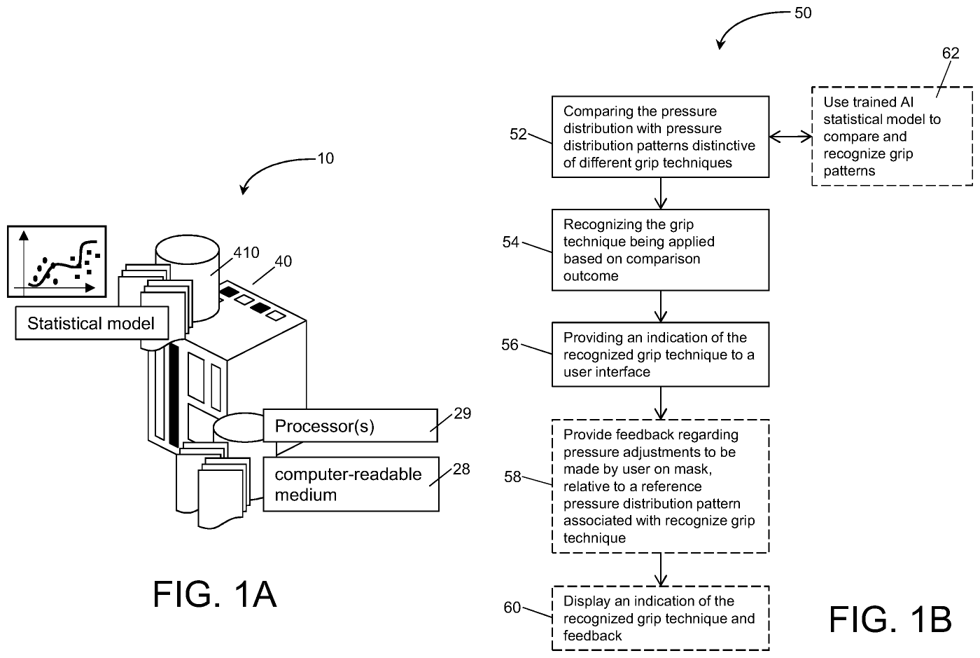

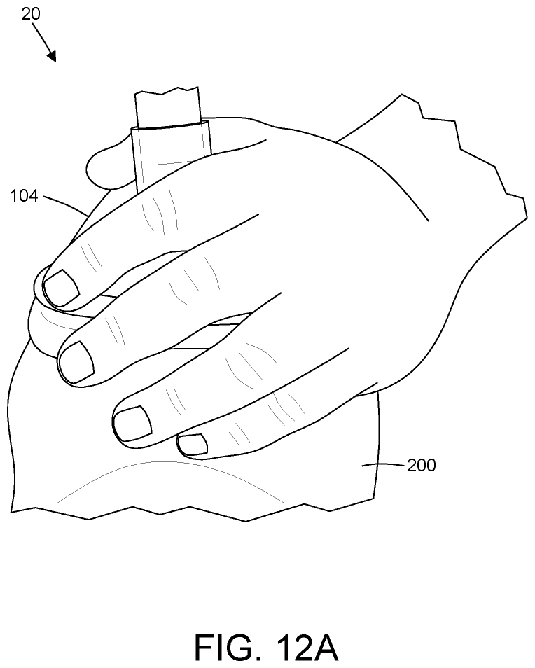

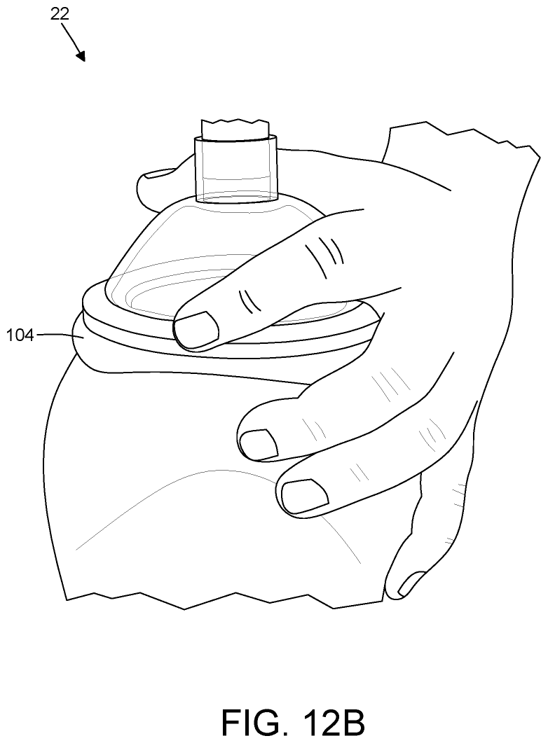

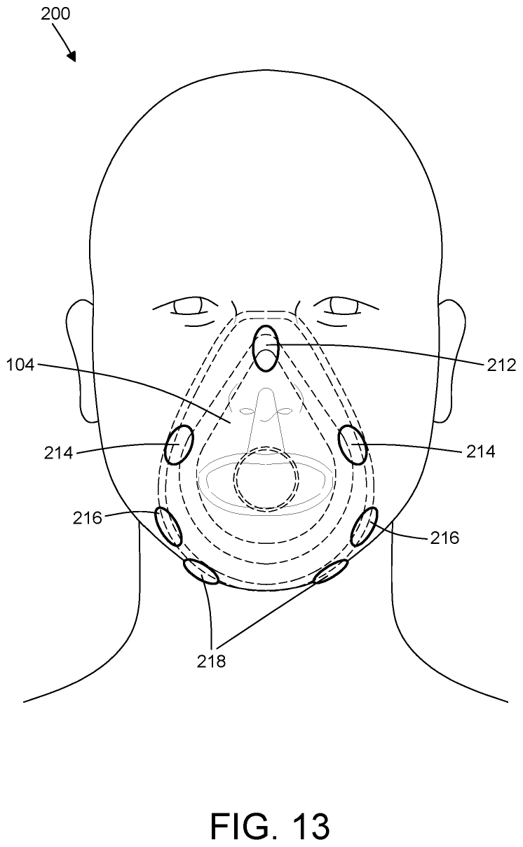

With reference to FIG. 1A, a system 10 is provided, for recognizing or detecting a grip technique being applied to the mask of bag-valve-mask, such as the face mask designed to sealingly engage with a face, around the nose and mouth. The face can be the face of a human or of an animal, or it can be the face of a manikin, used for training or evaluation purposes. The term “grip technique” is meant to refer to a position of the user's hand(s) on a mask, to maintain it in place during the ventilation process. For BVM masks, there are different grip techniques that can be used when sealing the face mask 104 on a face, including the “rotated hold” technique, shown in FIG. 12A, and the “C-E” technique, shown in FIG. 12B. The grip or grip technique can also be referred to as a sealing technique, where forces are applied on areas or regions around the periphery of the mask, to maximize the sealing of the mask on the face of an individual or mannequin. When positioning a face mask to seal it around the nose and mouth, the periphery of the mask will be subjected to different pressures there along, which can be captured and aggregated into a pressure distribution. Pressure distribution data captures how pressure is spread on a given surface or area, such as the mask periphery 105, or mask perimeter or edge. This pressure distribution can be used to characterize a grip technique being applied. The pressure distribution can be compared to previous recorded pressure distribution patterns, associated with different known grip techniques, to discriminate which grip technique is being applied. With the identification of the grip technique, better feedback may be provided to users in learning how to ventilate patients with a BVM. A pressure distribution pattern can correspond to a set of pressure data at discrete, predetermined points or regions along the periphery of the mask, or to a continuous distribution of pressures along the periphery. The pressure distribution pattern can be associated with a given grip technique or to a given expert, as examples only.

Referring to FIGS. 1A and 1B, the system 10 comprises a non-transitory computer-readable medium 28 (which may also be referred to as “non-volatile memory” or “non-volatile storage medium”), which can retain information for a given period, such as after power has been removed. Examples of non-transitory computer-readable medium includes ROM, magnetic computer storage devices, Solid State Drives (SSD), EPROM, flash memory storage, as examples only. The system 10 also comprises one or more processors 29, such as CPUs, DSPs and/or GPUs, to process the instructions stored in memory 28. The executable instructions stored in the non-transitory computer-readable medium 28 cause the one or more processors 29 to process pressure data, which is indicative of a measured pressure distribution applied on the face mask of a bag-valve-mask (step 52 in FIG. 1B) Pressure data signals are generated by the pressure transducers, which can detect the force applied on a specific surface region, typically at or near the periphery of the mask. The pressure transducers can be provided as individual devices, or in a continuous strip provided along the periphery of the mask. A pressure transducer can convert a physical force of pressure into a voltage output, such as 0-1V, or as an unamplified millivolt (mV) output. Capacitive, piezoelectric, or resistive transducers can be used for force measurements. Several pressure transducers can be used to detect the pressure applied on the mask at different locations, so as to generate the pressure distribution along the periphery of the mask. The combined pressure data signals generated by the set of transducers is used to derive the pressure distribution applied along a periphery around the nose and mouth at a given time, or for a given period. Pressure transducers can be part of pressure sensors or pressure sensing modules, which may include signal conditioning and communications electronics. In possible embodiments, the respective outputs of the plurality of pressure sensors connects to input ports, connectors or pins of a controller, such as a microcontroller, which can convert the voltage signals, indicative of the force applied, into pressure data, or pressure measurement. The pressure data can for instance be stored in V or mV, or converted to pressure measures in N, PSI, bar or other suitable unit.

The pressure distribution, which can correspond to a set or collection of pressure data, can be stored on the non-transitory computer-readable medium. The pressure data can be accessed, such as by receiving it from the medium or querying the medium, and compared to other pressure distribution patterns, distinctive of different grip techniques (step 52). In other words, the pressure distribution can be compared, or assessed, against one or more pressure distribution patterns, distinctive of different grip techniques. The outcome of the comparison is a determination or prediction of how close the measured pressure distribution is from a reference distribution pattern and/or how well is conforms to this reference pressure distribution pattern. It is appreciated that the comparison can but needs not be a one-by-one comparison of individual patterns. The comparison can for instance rely on a predictive, descriptive or decision model. Based on the comparison outcome the system 10 can recognize which one of the grip techniques is being applied (step 54) and provide an indication of the technique recognized (step 56).

In possible embodiments, a trained machine learning model, for instance a trained statistical model 410, can be used to perform the comparison and make the grip technique determination (step 62). A trained statistical model can be generated from learning algorithms that analyze the pressure distribution data for classification, for instance by performing regression analysis. The comparison outcome can allow the pressure distribution data to be classified into a given class, which can correspond to a grip technique, for instance based on a probability or likelihood associated with the outcome that the measured pressure distribution pertains to this grip technique class or on the result of a scoring function associated with the pressure distribution and this grip technique class. The indication of the grip technique recognized can be provided in real time or after the manipulations have been performed, off-line. It will be understood that the training of a statistical model is performed using historical pressure distribution patterns. When in used in production, one skilled in the art will understand that the statistical model 410 does not actively compare newly gathered pressure data with previously collected pressure distribution patterns, but that the comparing step is conducted via the trained configuration of the statistical model, said trained configuration reflecting the historical pressure distribution patterns, distinctive of different grip techniques.

The different pressure distribution patterns preferably correspond to known grip techniques, such as to the “rotated hold” or “football C-grip” technique 20 (FIG. 12A), the “C-E” technique 22 (FIG. 126) or the two-handed technique. It should be noted, however, that these grip techniques are exemplary, and that other grip or positioning techniques can be identified with the system 10. The system 10 may also be configured to detect new grip techniques being applied, using self-learning AI statistical models. The grip technique identified can be indicated using different outputs (step 60), including lights or sounds, or more conveniently, with text and/or icons, on a graphical user interface displayed on the display screen of a computer, tablet or smart phone. Optionally, the system can be configured and adapted to provide feedback to users on pressure adjustments to be made, relative to a reference pressure distribution pattern, associated with the identified grip technique used (step 58).

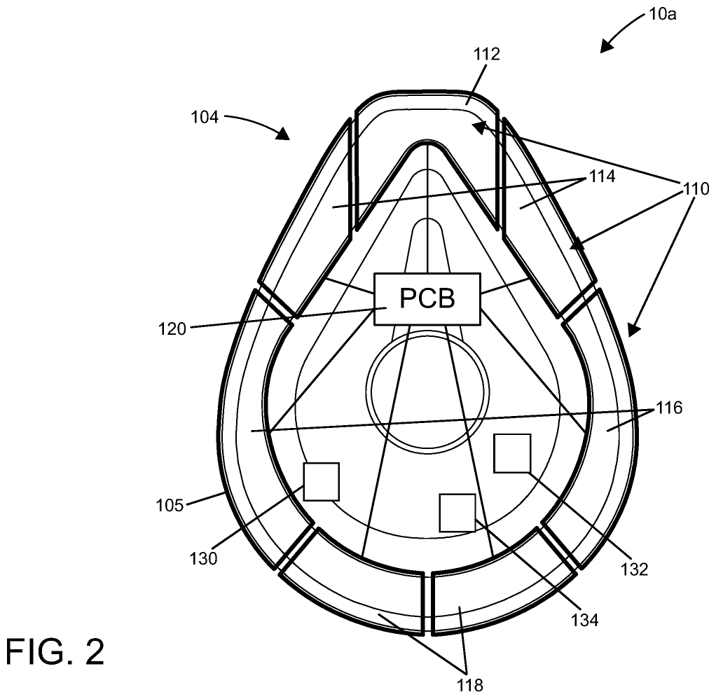

Referring now to FIG. 2, a possible embodiment of a system 10a is shown. This embodiment of the system 10a includes a face mask 104 of a BVM, and a PCB 120, which comprises storage and processing means. If the system 10a is to be used with real patients, the system 10a can include a complete and functional BVM, with the mask, airbag and valve components. However, for simulation or training purposes, the system 10a may solely include the mask 104, when the objective is mainly to detect grip techniques and/or provide feedback to users for training them to correctly position the mask for a given grip technique. The mask 104 has a periphery or edge 105, which is configured to engage a person's face around the nose and mouth. The mask 104 can include a plurality of pressure transducers, as well as position, barometric and/or airflow sensors, 130, 132, 134. The pressure sensors include the pressure transducers 110, which can convert the different pressure levels applied thereon into electrical signals, typically measured in voltage. The pressure sensors can be provided with conditioning and/or communication electronics, to process the electrical signals into digital pressure data signals and transmit the pressure data signals to other components, such as controllers or ports. This transmission from the pressure sensors to other devices can be made via a wired or a wireless connection (such as via Bluetooth.)

Pressure transducers can be positioned at different locations along the periphery of a portion of the face mask 104, such as in a sealing cuff bordering the mask. Pressure transducers may comprise a nose pressure transducer 112, left and right around nose pressure transducers 114, left and right cheek pressure transducers 116 and left and right chin pressure transducers 118. In the exemplary mask configuration, transducer 112 is positioned directly on a first peripheral portion of the mask 104 adapted to engage the mask proximate to the ridge of the nose, the second and third transducers 114 are adapted to engage the mask on either side of the nose, the fourth and fifth transducers 116 are adapted to engage the mask along the cheeks, and the sixth and seventh transducers 118 are adapted to engage the mask on either side of the chin. Other mask and/or pressure transducer configurations are possible. For instance, the mask 104 can have a chin portion extending therefrom, and some of the transducers can be provided on this chin portion.

Still referring to FIG. 2, the system may comprise one or more position sensor(s), such as position sensor 132, to collect position and/or orientation data, which can be used by the processing device, in combination with the pressure data, to identify the current grip technique being used. The position sensor 132 may for example include a Micro-Electro-Mechanical System (MEMS) chip that can provide positional and/or orientation data relative to a reference point. The position data, collected from the position sensor, can be combined with pressure data, to predict the grip technique being applied. The position sensor 132 may also be used to discriminate or distinguish periods during which data should be collected, such as when the mask is in position and sealed on the face, corresponding to stationary periods, from periods during which data should not be collected, such as when the mask is away from the face or manikin, or is being placed on or removed from the face—corresponding to transitional periods. Alternatively, the pressure data signals generated by the pressure transducers can be used to distinguish stationary from transitional periods, where during transitional periods, either no pressure or a pressure below a threshold configured to represent a typical pressure detected when the mask is being moved to a different position is detected. Since an objective is to identify grip techniques and to qualify the mask placement procedure during stationary periods, the system can be configured with instructions that can detect whether there are large pressure variations during continuous periods. For example, stationary periods can be detected by applying a standard deviation threshold on the pressure data signals generated during a given period, or by averaging the first derivative of the pressure data signals collected. Another possible way of identifying stationary periods consists in verifying whether the pressure applied on the mask is above a given threshold, over a given time period. The pressure data signals generated during stationary periods can thus be processed as described above, but data from the transitional periods can be discarded.

The system may also include one or more airflow sensor(s) 130, provided within the mask 104 to detect leaks when the mask is in place, and provide additional information on the efficiency of the seal of the mask, when applying a given grip technique. By comparing airflow data or signals, with other reference airflow data or signals, the system can determine if the current grip technique adequately seals the mask on the person's face, and the efficiency of the ventilation procedure. The airflow measurement data can be combined with the pressure distribution data to qualify how well a given grip technique is being applied, for example by outputting a score indicative of a degree of sealing of the mask on the face of a patient or mannequin for the detected grip.

Since pressure measurements can be sensitive to ambient pressure and temperature, the system may also include barometric and/or temperature sensors 134. The system's memory may include compensation algorithms to compensate for changes of temperature and altitude, according to the conditions in which the mask is being used. In other words, the pressure data can be adjusted based on barometric and/or temperature measures, by increasing or decreasing the pressure measures, as examples only. Temperature and atmospheric pressure data may also be accessed wirelessly, via web services for example.

Still referring to FIG. 2, according to this embodiment, the system 10a comprises a printed circuit board (PCB) 120. The PCB can be provided on the face mask, as in FIG. 2, or elsewhere on the BVM. The non-transitory computer-readable medium of the PCB can store the instructions for its processor to execute, to determine the grip technique being applied. The computer-readable medium can also store instructions to provide feedback on how well the grip technique is being applied, compared to a “reference” grip technique, characterized by a corresponding reference pressure distribution pattern. Lights can be provided near the edge of the mask 104 and be associated with corresponding pressure transducers, to provide visual feedback to users on their performance in applying a given grip technique. Lights of different colours, or blinking patterns, can be used as visual assessment indicators. For example, a light may be green if the pressure applied on a given region is adequate and red if the pressure is too high or too low, compared to a reference pattern associated with the identified grip technique. In preferred embodiments, this assessment is provided in real-time e.g., by being updated on a regular or continual basis as the pressure data changes over time. A speaker may also be provided on the mask, to indicate which grip technique has been detected and/or to provide audio feedback to the user when positioning or sealing the mask. A user interface can thus be integrated in the mask to provide feedback to users relating to the pressure adjustment needed along the periphery of the mask, including whether a proper pressure distribution is being applied to the mask. The lights, which can be LEDs, provide real-time feedback to the user, based on the pressure the user has applied. Trainees receive a visual feedback depending on whether or not they apply the right amount of pressure on a particular region of the mask, and if the pressure is inadequate, which region of the mask must receive more or less pressure.

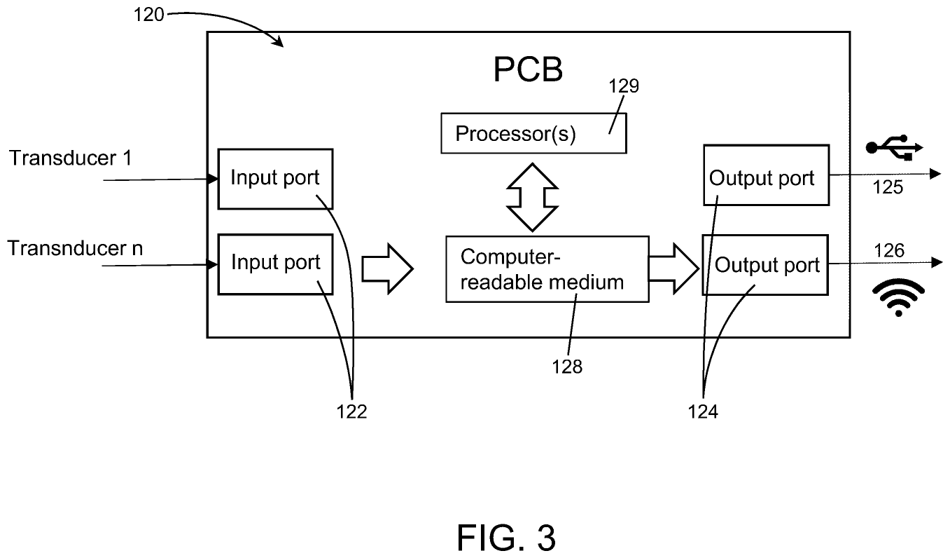

Referring to FIG. 3, the PCB 120 may include one or more input ports 122 or connections, for receiving wires or connectors from the transducers. The wires convey pressure data signals from the plurality of pressure transducers to input ports of controllers, as an example only. The signal conditioning, such as filtering, amplification, and conversion, may be performed by the PCB. The PCB can include one or more output ports 124 or connections, for sending pressure data and/or for sending the identification of the grip technique, or feedback indications, via a wired connection 125 or wireless connection 126. The processor 129 and the computer-readable medium 128 are mounted on the PCB 120, enabling the proposed method to be executed locally, within a single local processing device. Thus, in possible embodiments, the trained machine learning model e.g., the trained statistical model, can be stored and run from the PCB's microcontroller. The determination and assessment of the grip technique can thus be performed on the mask 104, and indicators (such as lights) may also be provided on the mask and controlled based on the determination made. In alternative embodiments, the one or more output ports 124 may also connect to other processing devices, as will be explained below.

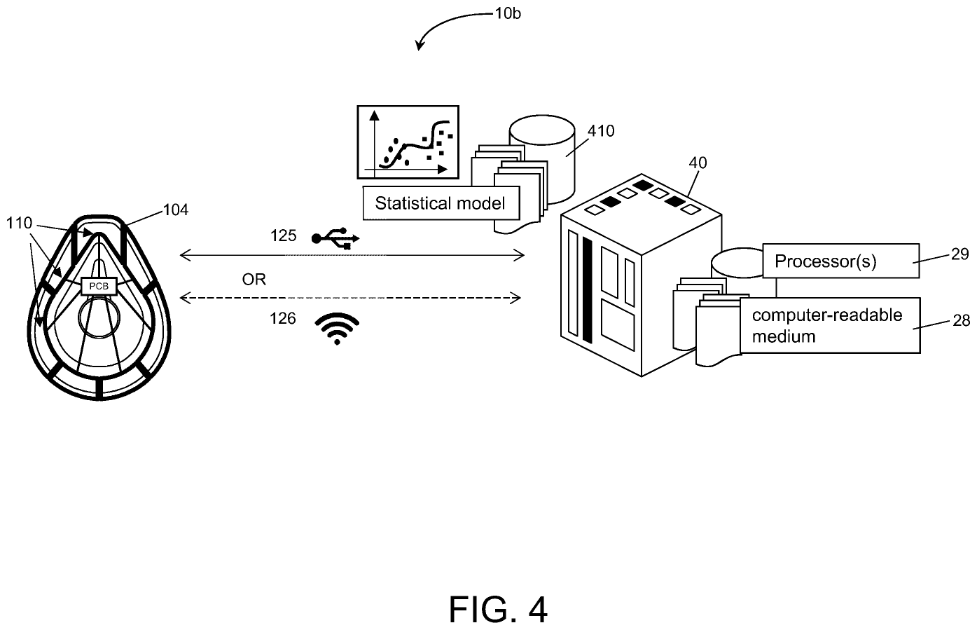

Referring now to FIG. 4, in another possible embodiment, processor(s) and computer-readable medium of the system can be located remotely from the instrumented face mask 104, for example, as part of another processing device 40. Like the system 10a of FIG. 2, the system 10b shown in FIG. 4 comprises a face mask 104, with an inflated cuff divided in multiple cuff portions, interconnected around the mask. Each cuff portion comprises a pressure transducer, the different pressure transducers 110 being connected to and communicating with components of a PCB, including a memory and a microcontroller which computes pressure changes based on readings of the different pressure transducers. Aggregating the pressure data from each individual transducer, the microcontroller can derive the pressure distribution around the mouth and nose when the mask is positioned, and pressure is applied thereon.

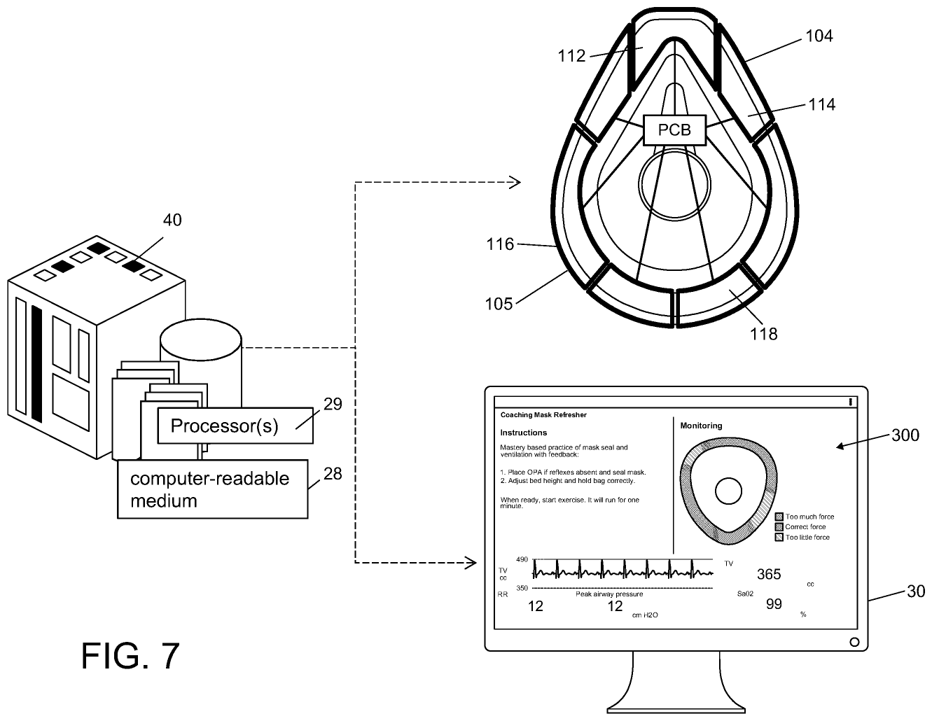

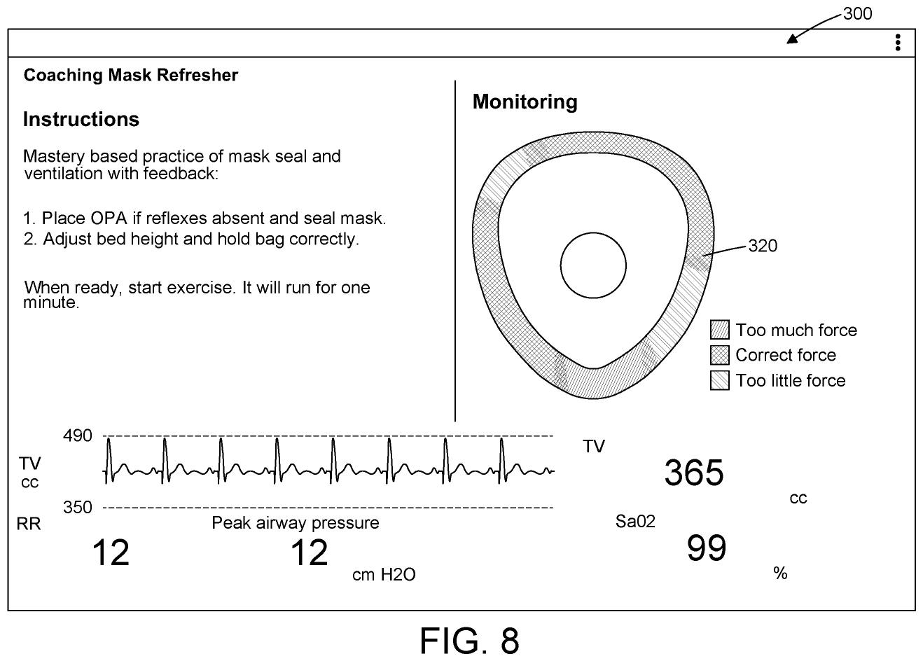

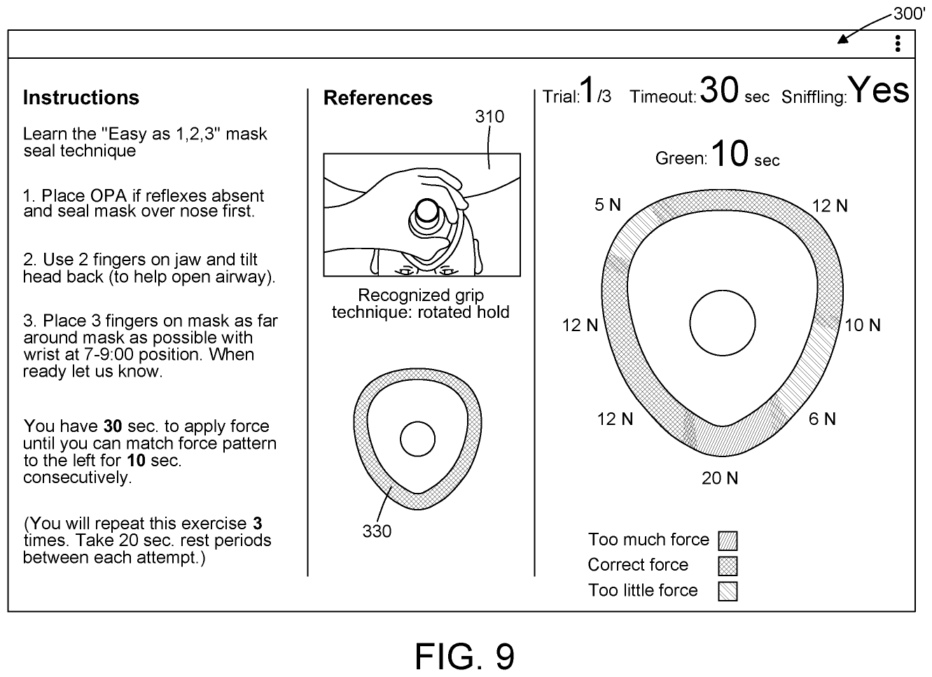

Still referring to FIG. 4, and also to FIG. 7, the measured pressure distribution data can be sent to the processing device 40, which can be a computer comprising a software application to display a graphical user interface 300 on a display screen 30, so as to show how the pressure distribution along the edge of the mask changes. The user interface can thus be a graphical user interface 300, including a visual representation of the pressure distribution along the periphery 105 of the mask 104. As shown in FIGS. 8 and 9, the pressure distribution can be represented in a graphical user interface 300 or 300′ with a variety of colours, icons, numbers, and letters to indicate the recognized grip technique and the user applied pressure on the mask. As an example, both figures display a mask with different colours indicating whether the user applied the right amount of pressure on a specific region of the mask, with green being a correct amount, red being too much and blue being too little. Other colours or means can be used to convey this information on the graphical user interface 300 or 300′. The graphical user interface 300 or 300′ may further display an indication as to how far or how close the measured pressure distribution is compared to a reference distribution pattern—using colours, text or numbers, indicative of a positioning score. The reference distribution pattern is associated with the identified grip. Therefore, during training, a user can visually determine if the pressure applied while placing or holding the mask is in accordance with the “reference” or “gold standard” distribution pattern for this grip technique—corresponding for example to the pressure an expert would apply. This assessment can be performed by feeding a trained machine learning model such as trained statistical model with pressure data collected and derived from the various transducers so that it can output a grip technique class, and possibly a grip score, indicative of how close the pressure data measured is from a reference grip pattern. The score can for instance be provided as a probability or likelihood or as the result of a scoring function used in a classifier. Similar to the light system described for the embodiment of FIG. 2, the graphical user interface 300 or 300′ can provide feedback to the user regarding pressure adjustments to be made along the periphery when positioning and holding the mask, for example by indicating on which portions of the mask too much, too little, or enough pressure is being applied. The graphical user interface 300 or 300′ can also include, as shown in FIGS. 8 and 9, instructions related to the recognized grip technique, metrics related to the performance of the user, or the operational status of the mask and/or manikin.

The system 10b can thus provide feedback to users or trainers, e.g., an appreciation (or indication) of how well the mask seal is being executed. The system 10b does so by comparing the distribution pattern to reference (or expert) distribution patterns to provide feedback to users, such as where on the mask they applied too much or too little pressure, for example, based on thresholds, or to indicate possible leak locations. For example, a reference lookup table can be stored in memory 28 and used to compare the pressure measured at the different sensing areas or regions along a periphery around the mouth and nose, to pre-established reference pressure thresholds for each pressure transducer, or sensing area. Based on said comparison, the system can, in addition to identifying the grip technique being applied, provide feedback on the graphical user interface 300, by indicating how far the pressure data in a given area is from the reference pressure data, and/or by indicating what needs to be done to get closer to the reference pressure. In preferred embodiments, the feedback can be provided in real-time e.g., by being updated on a regular or continual basis as the pressure data changes over time.

Instead of lookup tables, a statistical model 410 can be used. Sessions with expert user(s) can be conducted, during which “reference” or “expert” pressure distribution patterns are stored and labelled for instance based on the grip technique and/or based on the expertise level. The statistical model 410 can be trained using the labelled pressure distribution patterns, such that when presented with newly gathered pressure distribution data collected while novice users are being trained, the trained statistical model 410 can predict and thus recognize which grip technique is being used, and also preferably, indicate how well the grip technique is being applied, for example with a performance score. This information can further help trainees improve their skills by proposing personalized tips on the graphical use interface 300 or 300′, as shown in FIGS. 8 and 9, where a visual representation of the measured pressure distribution 320 is shown, along with the identified grip technique 310, and a representation of a reference pressure distribution 330. Alternatively, unsupervised predictive models can be used to predict and thus recognize or identify the different grip techniques, where each unsupervised predictive model is associated with a distinct grip technique.

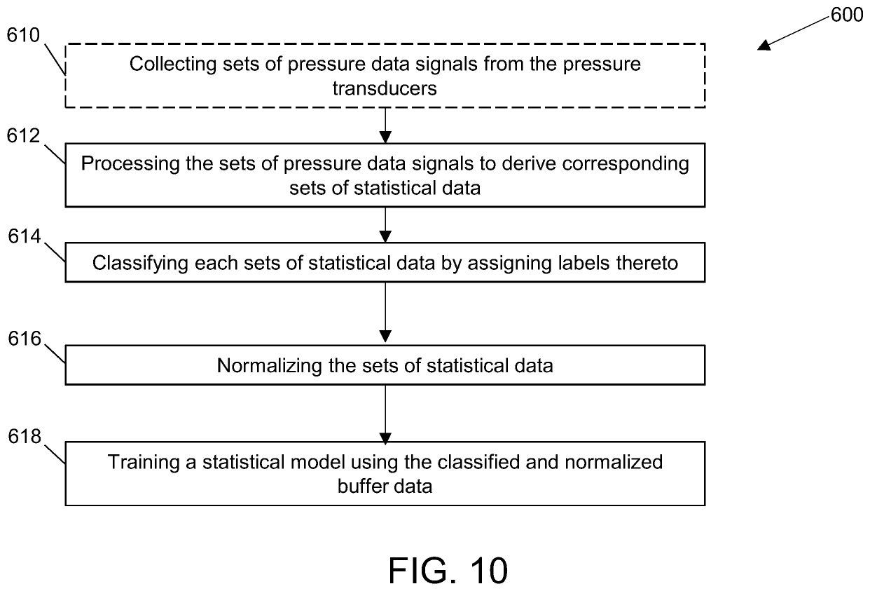

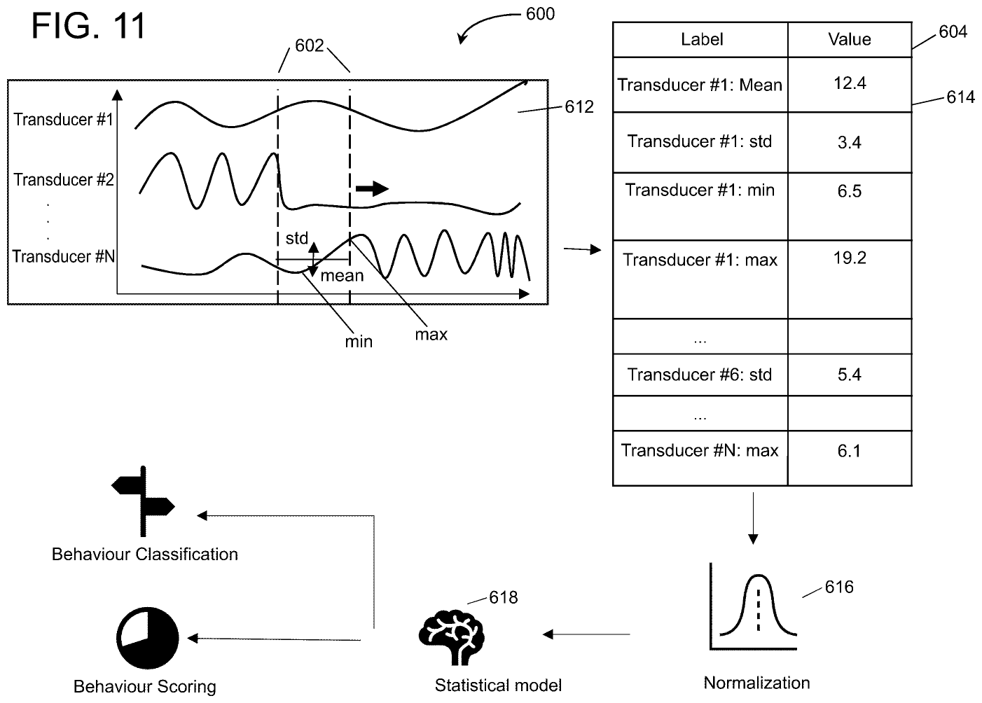

Referring now to FIGS. 10 and 11, a method 600 will be described, to configure or train the systems 10 previously presented. Sets of pressure data signals are collected (step 610) from the transducers numbered 1 to N, as illustrated in the graph of FIG. 11. The pressure data signal can be converted into time series and processed as such. The sets of pressure data signals are indicative of a distinct pressure distribution applied along the periphery 105 of the mask 104. The sets of pressure data signals, which can be kept e.g., as voltages, or converted into pressure data and/or normalized pressure data (such as from 0 to 1) are allocated in time buffers 602 (step 612). The time buffers may overlap or not. A time buffer can thus be considered as a data structure, holding pressure data signals generated during a given period. The sets of pressure data signals are processed to derive corresponding sets of statistical data. As in FIG. 11, time buffer 602 corresponds to a period during which several pressure data signal readings can be collected from transducers 1 to N. The pressure data signals are collected either continuously or periodically, for instance according to the transducer and/or PCB sampling frequency. The duration of the time buffer can be configurable but will typically correspond to the refresh rate of the transducers, where applicable, and may also depend on the computing capabilities of the processor(s) and other hardware components. Typically, the pressure transducers operate on a same frequency, where applicable, and preferably the processing chip is sufficiently powerful to process a collected dataset within a same cycle. Otherwise, the processing chip can sample at a lower frequency as long as the real-time effect is preserved in embodiments where the indicator is expected in real time. For each set of pressure data signals, statistical data, including for example the average, standard deviation, coefficient of variation, minimal and maximal pressure, voltage or normalized value, is computed and stored in the non-transitory computer-readable memory, for example in a table, array or matrix 604, as illustrated in FIG. 11. Each set of statistical data is then classified, and a label is assigned thereto (step 614). The labels can correspond to the grip technique being applied, and/or as to whether the pressure along the periphery 105 of the mask 104 is properly distributed, for a given grip technique. In possible embodiments, the expert applying the mask determines the appropriate label to be assigned on the basis of his or her training, and labels assigned to sets of statistical data is based on this determination. For example, a first label can be associated with a grip technique being applied. Another label can be associated with an expert having conducted a mask installations/donning, and from which pressure distribution data has been used to train the machine learning model. Another label can correspond to whether the mask is correctly positioned, based on scale. The statistical data derived from time series of pressure distribution data is then normalized (step 616), and the statistical model can be trained, using the classified and normalized statistical data, to recognize whether captured pressure data correspond to a mask 104 being positioned with a valid pressure distribution along its periphery 105, for a given grip technique (step 618). Different statistical models can be used, such as for instance a support vector machine model, a relevance vector machine model or a neural network model. The statistical model can be trained to detect the different grip techniques, and to assign different categories to the different pressure distribution, such as good, acceptable or bad, with a percentage or a score on a given scale. Different weights can be applied to the different categories, depending on the desired behaviour for the system. For example, in some embodiments, a given class or category, such as the “good” category can be configured to be more difficult to attain, by adjusting weights of the learning model accordingly.

Once trained, the statistical model can be presented with newly gathered sets of pressure distribution data, to find similarities with predetermined grip techniques, i.e., a probability that the technique being applied corresponds to a given reference grip technique. In possible embodiments, the statistical model can be used to assess any pressure dataset regardless of the time of capture. For instance, pressure data to be tested can be collected prior to collecting the training data, and before generating the trained statistical model. The generated model would then be tested using the testing data, collected prior to the generation of the model.

Alternatively, instead of planning sessions with experts to gather pressure data, pressure distribution data signals can be gathered over various sessions. The statistical model can be configured or trained to group similar pressure distribution patterns, and to generate a list of most frequently used pressure distribution patterns. The group of distribution patterns having the best data, e.g., for which no leak has been detected, or presenting a uniform pressure distribution pattern over the entire periphery of the mask, can be classified as “good” and can be selected as the “expert” or “gold standard” reference. Although the preferred machine learning model for this embodiment is a support vector machine model, it is possible to use other types of supervised machine learning algorithms, such as relevance vector machine model or a neural network model.

In yet other possible embodiments of the system, a software application can be configured and adapted to track the evolution of the performance of trainees in applying a given grip technique. The software application can use the results outputted by the statistical model to do so, for example by tracking or monitoring the performance score of users over time. The system can be configured to display the different scores of a user over time, allowing users to quantify the required practice needed to attain an expert standard for a particular grip technique. Given that the statistical model allows grouping similar pressure distribution patterns, the system can determine how many recurring patterns are performed by novices and how training can be improved to reach a proper grip technique. As illustrated in FIGS. 8 and 9, the system can be configured to provide personalized guidance, based on recurring errors detected by the statistical model. Performance ratings for each category can be determined by applying a scaler during training of the statistical model, such that a probability is outputted, or such that a score (on a scale of 1 to 10, for example) is derived from this probability. The calibration of the probability output by the statistical model can be performed using a Platt scaling, as an example only, which transforms a classification output into a probability, as illustrated in the lower left side of FIG. 11, where either one of a classification or a score is outputted by the statistical model. A person skilled in the art will appreciate that alternative ways of obtaining a probability can be used, for instance by using a probabilistic classifier such as a relevance vector machine.

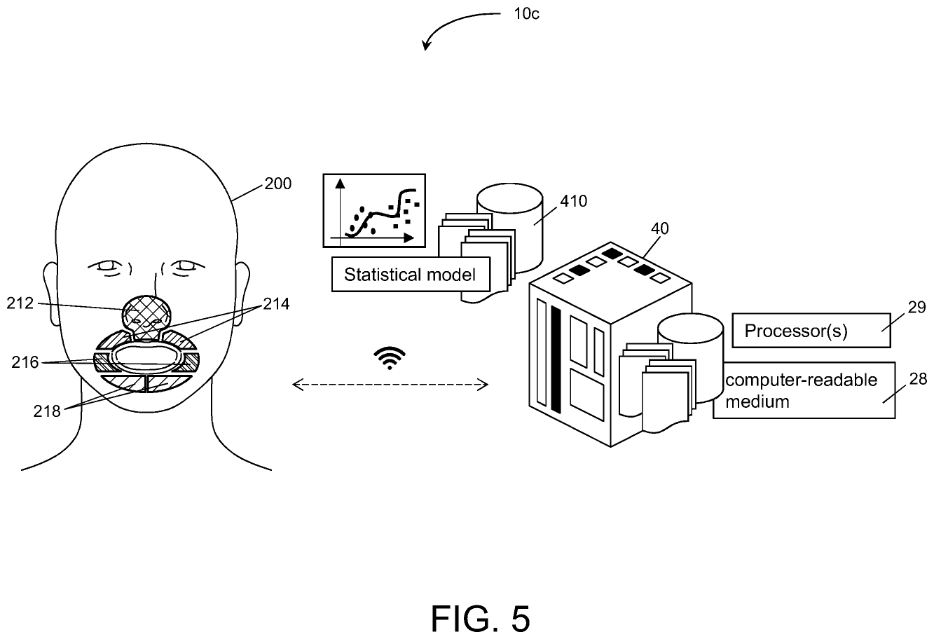

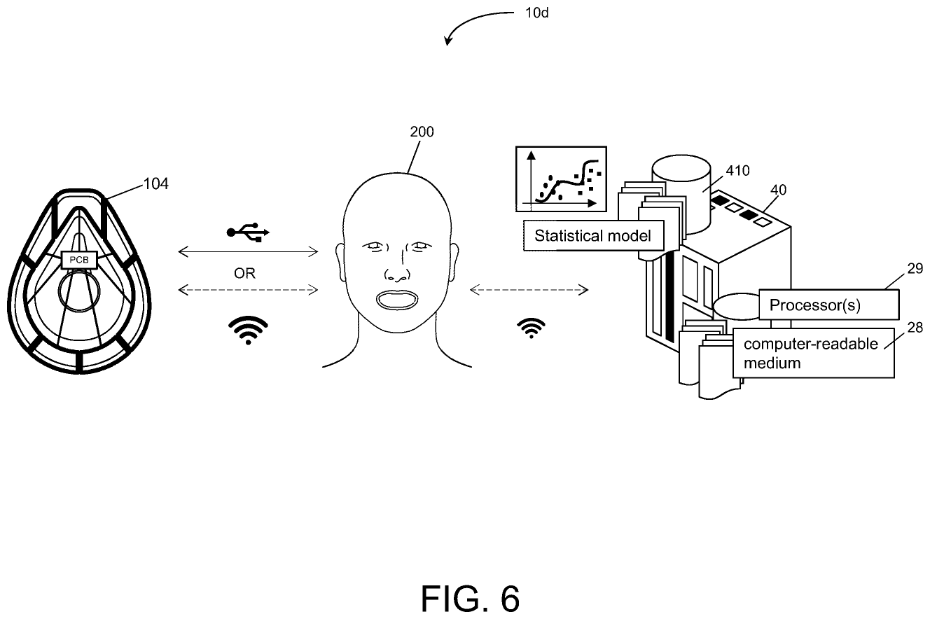

Referring now to FIGS. 5 and 6, other possible system embodiments 10c and 10d are shown. In the embodiment illustrated in FIG. 5, the pressure transducers 212, 214, 216 and 218 are provided on a training manikin 200, instead of being provided on the mask. The pressure transducers can transmit the pressure data signals via a wired or a wireless connection, to a processing device 40, which can process the pressure data signals, to derive the pressure distribution. This alternative is advantageous since the mask installation procedure can be taught using regular/non-instrumented BVMs. Alternatively, the manikin 200 can be provided with a PCB similar to the one described above, such that a portion of pressure data processing is performed by the PCB, before being sent to a dedicated software application run by the processing device 40. A statistical model 410 can also be used with this embodiment 10c.

FIG. 6 shows yet another possible system embodiment 10d, where the pressure transducers 110 are provided in a face mask 104, but where the training manikin 200 acts as a hub, including processing and data storage capacities. The training manikin 200 receives and processes the pressure data signals generated by the pressure transducers, and sends the processed pressure data signals, i.e., the pressure distribution, to the processing device 40, to feed the statistical model therewith. The processing device 40 can display a graphical user interface with the pressure distribution, an indication of the grip technique recognized and, optionally, indications to the users as to where pressure should be increased or lowered on the face mask to ensure proper seal and ventilation.

It will be appreciated from the foregoing disclosure that the system described provides for the recognition, detection or identification of different grip techniques; can help trainees or professionals to improve their grip technique when positioning and using a bag-valve-mask for the ventilation of airways; and can help users achieve a good grip technique in possibly less time, since feedback can be provided and is adapted based on the grip technique identified. The system can advantageously take different configurations, such that the pressure data processing can be performed, at least in part, remotely from the users, if needed. Training of the system may also be done based only on previously collected pressure data. The proposed system and method can also provide relevant comparative data between trainees and expert clinicians, which can be used to determine how often evaluations should be performed, for maintaining or improving the proper BVM grip techniques. The system and method can also be used by trained practitioners in real-life situations.

While the invention has been described in conjunction with the exemplary embodiment described above, many equivalent modifications and variations will be apparent to those skilled in the art when given this disclosure. Accordingly, the exemplary embodiment set forth above is considered to be illustrative and not limiting. The scope of the claims should not be limited by the preferred embodiment set forth in this disclosure but should be given the broadest interpretation consistent with the description as a whole.

1. A system for identifying a grip technique being applied by a user to a face mask for sealingly engaging a face along a mask periphery around the nose and mouth, the system comprising:
a processor and a non-transitory computer-readable medium having stored thereon processor-executable instructions for:
accessing pressure data indicative of a pressure distribution applied along the mask periphery, the pressure distribution being applied by the user for sealing the face mask on the face;
comparing the pressure data with pressure distribution pattern data distinctive of different grip techniques;
detecting which of the different grip techniques is being applied on the basis of an outcome of the comparing; and
providing an indication of the grip technique detected to a user interface.

2. The system of claim 1, wherein the non-transitory computer-readable medium further has stored thereon processor-executable instructions for:
generating feedback regarding pressure adjustments to be made for the proper application of the detected grip technique; and
providing the feedback to the user interface.

3. The system of claim 2, wherein providing the feedback is performed in real time while the face mask is being positioned.

4. The system of claim 3, wherein the user interface comprises a display and providing the indication of the grip technique is performed through a graphical user interface presented within the display.

5. The system of claim 1, wherein the user interface comprises a visual representation of the pressure distribution applied along the periphery of the face mask.

6. The system of claim 1, wherein the outcome is associated with a probability or likelihood that the pressure distribution applied is one of the pressure distribution patterns distinctive of the detected grip technique.

7. The system of claim 1, wherein the non-transitory computer-readable medium comprises a trained machine learning model, and wherein detecting a given one of the different grip techniques comprises predicting the grip technique applied using the trained machine learning model.

8. The system of claim 7, wherein the trained machine learning model is configured to output a performance score indicative of the closeness of the pressure distribution applied on the mask from the pressure distribution pattern associated with the grip technique detected.

9. The system of claim 7, wherein the trained machine learning model is trained for assigning the pressure data to one of: an E-C grip technique, a two-hand grip technique and a rotated hold technique.

10. The system of claim 6, wherein the non-transitory computer-readable medium further has stored thereon processor-executable instructions for generating statistical data from the pressure data, the statistical data including mean, standard deviation, minimum and maximum values for time buffers, and the comparing is based on the statistical data.

11. The system of claim 1, wherein the user interface further comprises an indication of how close the pressure distribution applied is from the pressure distribution pattern distinctive of the detected grip technique.

12. The system of claim 1, wherein the non-transitory computer-readable medium further comprises processor-executable instructions for monitoring a performance of users in applying a given grip technique, by storing pressure data over time and associated performance scores assigned to the users.

13. The system of claim 1, further comprising pressure transducers configured to generate pressure data signals from which the pressure data is derived.

14. The system of claim 13, further comprising a printed circuit board (PCB) provided on the face mask, the PCB comprising:
input ports for collecting the pressure data signals from the pressure transducers; and
output ports for sending the pressure data and/or the indication of the grip technique detected via a wired or wireless connection to the computer-readable medium.

15. The system of claim 13, further comprising a position sensor provided on the mask for generating position data signals, and wherein the detecting also takes into account the position data signals.

16. The system of claim 1, wherein the non-transitory computer-readable medium comprises processor-executable instructions for detecting, from the pressure data, transitional periods corresponding to periods during which the face mask is being placed, removed or changed in position, and wherein the filtering is performed to prevent the comparing from being applied to portions of the pressure data generated during the transitional periods.

17. The system of claim 1, further comprising a barometric and/or a temperature sensor, wherein the pressure data are adjusted as a function of ambient temperature and/or ambient pressure data generated as the grip technique is being applied, whereby the comparing takes into consideration ambient conditions.

18. The system of claim 1, further comprising airflow sensors for generating airflow data signals within the mask portion, wherein the non-transitory computer-readable medium further comprises processor-executable instructions for detecting leaks by comparing the airflow data signals with reference air flow data associated with an adequate reference sealing engagement of the face mask on the face.

19. A computer-implemented method for identifying a grip technique being applied to a face mask by a user for sealingly engaging a face along a periphery around the nose and mouth, the method comprising:
comparing pressure data indicative of a pressure distribution applied by the user along the periphery for sealing the face mask on the face, with distribution patterns distinctive of different grip techniques;
detecting which of the grip techniques is being applied on the basis of an outcome of the comparing; and
providing an indication of the detected grip technique to a user interface.

20. A non-transitory computer-readable medium having stored thereon processor-executable instructions for identifying a grip technique being applied to a face mask by a user for sealingly engaging a face along a perimeter around the nose and mouth, the instructions causing one or more processors to:
compare pressure data indicative of a pressure distribution applied by the user along the periphery with distribution patterns distinctive of different grip techniques;
detecting which of the grip techniques is being applied on the basis of an outcome of the comparing; and
provide an indication of the detected grip technique to a user interface.
